(19) United States
(12) Patent Application Publication
(10) Pub. No.: US 2024/0238506 A1
(43) Pub. Date: Jul. 18, 2024

(54) Drip Chamber for a Fluid Administration System

(71) Applicant: Fresenius Kabi Deutschland GmbH, Bad Homburg (DE)

(72) Inventor: Steffen Baumgart, Hünfeld (DE)

(21) Appl. No.: 18/549,849

(22) PCT Filed: Mar. 16, 2022
(86) PCT No.: PCT/EP2022/056838

§ 371 Date: Sep. 8, 2023

(30) Foreign Application Priority Data
May 3, 2021 (EP) 21171896.0

(51) Int. Cl. A61M 5/14

(52) U.S. Cl. CPC A61M 5/1411; A61M 2205/75

(57) ABSTRACT

A drip chamber for a fluid administration system, comprising: a reservoir (3, 3′) comprising an inlet orifice (5) at a top end region (13A) for inserting a fluid, and an outlet orifice (7, 7′) at a bottom end region (13B), wherein the bottom end region (13B) is located in gravitational direction opposite and below the top end region (13A), wherein a plunger element (11, 11′) moveably arranged within the reservoir (3, 3′) between a first position in the top end region (13A) and a second position in the bottom end region (13B), wherein the plunger element (11, 11′) comprises a fluid conducting channel (15) for regulating the flow of fluid from the inside of the reservoir (3, 3′) to the outlet orifice (7, 7′) when the plunger element (11, 11′) is located in the second position.

The invention relates to a drip chamber according to the preamble of claim 1, and to a method to operate a drip chamber.

A drip chamber of this kind comprises a reservoir comprising an inlet orifice at a top end region for inserting a fluid, and an outlet orifice at a bottom end region, wherein the bottom end region is located in gravitational direction opposite and below the top end region.

Such a drip chamber can generally be used in a fluid administration system to administer fluids to a location within a patient's body, such as for intravenous, arterial, intravascular, peritoneal, or non-vascular administration of fluid.

One common technique of administering fluids into a patient's blood flow is through an intravenous, IV, fluid administration system, which is also often referred to as IV infusion set such as the one described in EP 3 695 860 A1. Here, a fluid bag is connected to the top end region of the drip chamber and a tube, through which the fluid flows to the patient, is connected to the bottom end region of the drip chamber. The IV fluid administration system can be, for example, arranged at a pole at the bedside of the patient.

Most prior art drip chambers used for administering fluids are comprised of multiple parts. One part might comprise a hard material forming a spike-like structure arranged at the inlet orifice for puncturing the fluid bag to allow fluid flowing towards the inside of the reservoir, whereas the reservoir might comprise a soft material to allow squeezing of the reservoir for priming. The term “priming” is commonly used in the prior art to refer to pumping fluid into the reservoir of the drip chamber. The drip chamber allows gas such as air residues, also often referred to as air bubbles, to rise out from the fluid so that gas is not passed downstream to the tubes, which access a patient's blood flow. Specifically, if air bubbles are allowed to enter a patient's blood stream while receiving the intravenous administration of liquids, the air bubbles can form an air embolism and cause serious injury to a patient.

In order to control the flow rate of the fluid out of the drip chamber to the patient, a clamp which is often realized as a roller clamp, can be placed on the tube and can be adjusted to control the fluid flow by squeezing the tube.

The known drip chamber requires multiple pumping making priming a rather cumbersome and imprecise process. When the known drip chamber is used in a fluid administration system together with a commonly used flow regulator such as a roller clamp, the system may be cost effective, but regulating the fluid flow cannot be achieved with high precision. Even though precision flow regulators for gravity infusion are known in the prior art, these flow regulators have a rather complex construction and need to be mounted inline via additional gluing connections.

It is an object of the instant invention to provide a drip chamber for a fluid administration system that allows easy assembly, uses fewer parts and allows precise control of the fluid flow.

This object is achieved by means of the drip chamber for a fluid administration system comprising the features of claim 1.

Herein, the invention relates to a drip chamber for a fluid administration system, comprising: a reservoir comprising an inlet orifice at a top end region for inserting a fluid, and an outlet orifice at a bottom end region, wherein the bottom end region is located in gravitational direction opposite and below the top end region, wherein a plunger element is moveably arranged within the reservoir between a first position in the top end region and a second position in the bottom end region, wherein the plunger element comprises a fluid conducting channel for regulating the flow of fluid from the inside of the reservoir to the outlet orifice when the plunger element is located in the second position.

The reservoir can have a spherical, paraboloid or cylindrical shape. Here, the reservoir could have the shape of a tube having a cylindrical shape with a substantially constant circular cross section along its length. The terms “top end region” and “bottom end region” can be used herein to refer to opposite end sections of the reservoir. The term “gravitational direction” can be used herein to define that a “gravitational acceleration” can be the fluid feeding driving force from the inlet orifice to the outlet orifice.

The inlet orifice at the top end could be an opening to which a fluid bag that can be also referred to as a fluid container, holding the fluid can be directly or indirectly connected. For example, a spike-like structure could be arranged at the inlet orifice facing away from the reservoir for puncturing the fluid bag when the drip chamber is brought into contact with the fluid bag so that the fluid can flow into the reservoir.

The outlet orifice at the bottom end can be arranged in the sidewall of the reservoir.

The plunger element which can be also referred to as piston can be adapted to fit tightly within the reservoir and can be inserted into the reservoir through an end of the reservoir at the bottom end section that can be open and which is located opposite to an end of the reservoir at the top end section which comprises the inlet orifice.

For example, the plunger element can be linearly pulled and pushed along the inside of the reservoir between the first position and the second position.

The first position in the top end region can be understood as a position where the plunger element is closer to the top end of the reservoir than to the bottom end, where it abuts, or nearly abuts, against a wall of the reservoir which could be perpendicular to the sidewall and which comprises the inlet orifice. The second position in the bottom end region can be understood as a position where the plunger element is closer to the bottom end of the reservoir than to the top end, where it is at least partially aligned with the outlet orifice of the reservoir.

For example, when the plunger element is located in the first position in the top end region, and is then linearly pulled downwards towards the second position, the reservoir can take in fluid. Hence, pulling downwards the plunger element may create a suction, which can also be referred to as negative pressure, to prime the drip chamber.

Advantageously, the syringe like priming reduces multiple pumping from the user as it would be the case with priming a prior art drip chamber. In addition, since pumping by repeatedly squeezing the drip chamber is not necessary any longer, the entire drip-chamber can be made from one type of material, for example, from a rigid plastic material.

Also, the plunger element comprises a fluid conducting channel for regulating the flow of fluid from the inside of the reservoir to the outlet orifice when the plunger element is located in the second position.

For example, the plunger element could have an essentially U-shaped cross-section having a sidewall extending parallel to the sidewall of the reservoir and that fits tightly within the sidewall of the reservoir. The plunger element can have a disc-shaped structure at a lower surface where the driving member or handle can be attached to and which prevents fluid leaking from the open bottom end of the reservoir into which the plunger element can be inserted. The fluid conducting channel could be an opening in the sidewall of the U-shaped cross-section that when aligned with the outlet orifice of the reservoir in the sidewall of the reservoir allows fluid flowing from the inside of the reservoir to the outside of the reservoir via the outlet orifice. Depending on the position of the plunger element inside the reservoir, the passage to the outlet orifice could be closed, could be partially opened, or could be fully opened.

In an alternative example, the plunger element could be made in the shape of a disc that fights tightly within the sidewall of the reservoir and that comprises the fluid conducting channel extending through the material of the plunger element to allow fluid flowing from the inside of the reservoir to the outlet orifice.

Therefore, the drip chamber according to the invention allows easier priming and precisely regulating the flow of fluid leaving the drip chamber, making further flow regulators arranged downstream at the tube, through which the fluid flows from the drip chamber to the patient, obsolete.

Advantageously, the drip chamber according to the invention is easy to assemble comprises a minimum amount of components, while allowing to precisely control the flow exiting the drip chamber and easy one-step priming of the drip chamber.

In an example, the fluid conducting channel extends through a material of the plunger element, and comprises an inlet port opening into the reservoir and an outlet port opening at least partially into the outlet orifice when the outlet port is at least partially aligned with the outlet orifice in the second position.

Here, the channel can extend through a sidewall of an U-shaped plunger element as described above, or could extend through a solid material of the plunger element creating a fluid connection between the inside of the reservoir to the outlet port opening. For example, the channel could be tapered towards the outlet opening, where the inlet opening is funnel shaped towards the outlet opening.

In an example, the outlet port comprises an elongated opening with an increasing width, preferably gradually increasing or stepwise increasing, along the direction of its extension, and wherein the elongated opening extends at least partly around a circumferential surface of the plunger element. Also, according to this example, the outlet orifice comprises a circular opening, or an opening with an increasing width, preferably gradually increasing or stepwise increasing, along the direction of its extension.

Here, the elongated opening of the outlet port could, when seen at the circumferential surface, have the shape of a wedge. Hence, when the plunger element is arranged in the second position and then rotated inside the reservoir, rotating the plunger element can regulate the flow of fluid from inside the reservoir to the outside of the reservoir.

In an alternative example, the outlet port comprises an opening with a constant width along the direction of its extension, and wherein the opening extends at least partly around the circumferential surface of the plunger element. Also, according to this example, the outlet orifice comprises a circular opening, or an opening with an increasing width, preferably gradually increasing or stepwise increasing, along the direction of its extension.

Here, the flow of fluid can be regulated by linearly moving the plunger element up and down when arranged in the second position.

In another example, the outlet orifice is arranged in a sidewall of the reservoir. In an example, the outlet orifice comprises a nozzle for connecting a tube to the outlet orifice. For example, the tube through which the fluid flows to the patient can be connected to the nozzle. In an example, the nozzle comprises a bent section at a distal end, preferably a ninety degree bent section. Advantageously, the bent section could run at least partly parallel to the reservoir and allows an easy flow of fluid flow from the outlet orifice towards the patient.

In an example, the plunger element comprises a further fluid conducting channel comprising a further inlet port opening into the outlet orifice and a further outlet port opening into a surface of the plunger element directed towards the outside of the reservoir, wherein the outlet orifice is adapted to direct the fluid from the fluid conducting channel to the further fluid conducting channel, and wherein at least one of the outlet port of the fluid conducting channel, and/or the further inlet port of the further fluid conducting channel comprises an elongated opening with an increasing width, preferably gradually increasing or stepwise increasing, along the direction of its extension, and wherein the elongated opening extends at least partly around a circumferential surface of the plunger element.

Here, the tube, through which the fluid flows to the patient, can be advantageously connected to the further outlet port that can be located at the plunger element.

In an example, the plunger element comprises a sealing member encircling a lateral surface of the plunger element for providing a fluid-tight seal with the sidewall of the reservoir.

For example, the seal could comprise a rubber material that can be used to improve the sealing between the plunger element and the sidewall.

In another example, the plunger element comprises a driving member, preferably a handle, extending through the bottom end region for manually or automatically moving the plunger element within the reservoir.

The driving member could be essentially similar to the driving member used commonly for driving the plunger of a syringe and that can be operated manually or by means of an infusion pump. Alternatively, the driving member could have a large loop-shaped handle at the end opposite where it is attached to the plunger element, so that a user can extend a finger through the loop-shaped handle to easily move the plunger element inside the reservoir.

In an example, the plunger element comprises a fluid filter, the fluid filter can be arranged, in fluid direction, before the fluid conducting channel, or inside the fluid conducting channel. Here, the fluid filter can advantageously prevent infectious agents from entering the patient with the fluid, such as for example undiluted or undissolved drugs or particles of plastic or steel from administration equipment.

According to one embodiment a fluid filter is arranged between the top end region and the bottom end region. In an example, the fluid filter spans the opening of the plunger element with an U-shaped cross-section. Here the fluid filter is quite large compared to a filter inserted in the conducting channel. Thus, the likelihood of particle clogging the entire filter is reduced.

In an example, the plunger element is adapted to move:
(i) rotationally, around its own axis within the reservoir, and/or
(ii) vertically, along a central axis of the reservoir.

Essentially the plunger element can glide up and down within the sidewall of the reservoir, while also being rotated around its own axis.

The invention is also concerned with a method to operate a drip chamber for a fluid administration system, preferably a drip chamber according to one of the preceding claims, comprising:
connecting a fluid container comprising a fluid to an inlet orifice at a top end region of a reservoir to allow a fluid stream inside the reservoir, wherein the reservoir further comprises an outlet orifice at a bottom end region, wherein the bottom end region is located in gravitational direction opposite and below the top end region, and
moving a plunger element within the reservoir from a first position in the top end region to a second position in the bottom end region of the reservoir, wherein the plunger element comprises a fluid conducting channel for regulating the flow of fluid from the inside of the reservoir to the outlet orifice when the plunger element is located in the second position.

In an example, the method comprises moving the plunger element vertically along a central axis of the reservoir from the first position to the second position for priming the drip chamber.

In an example, the method further comprises: aligning, in the second position, an outlet port of the fluid conducting channel extending through a material of the plunger element with the outlet orifice to at least partly open a passage for fluid from an inlet port of the channel opening into the reservoir via the outlet port of the channel to the outlet orifice.

In an example, the aligning further comprises:
regulating a flow rate of the fluid through the outlet port by:
   (i) rotating the plunger element around its own axis, and/or
   (ii) vertically moving the plunger element along the central axis of the reservoir.

The idea underlying the invention shall subsequently be described in more detail with reference to the embodiments shown in the figures. Herein:

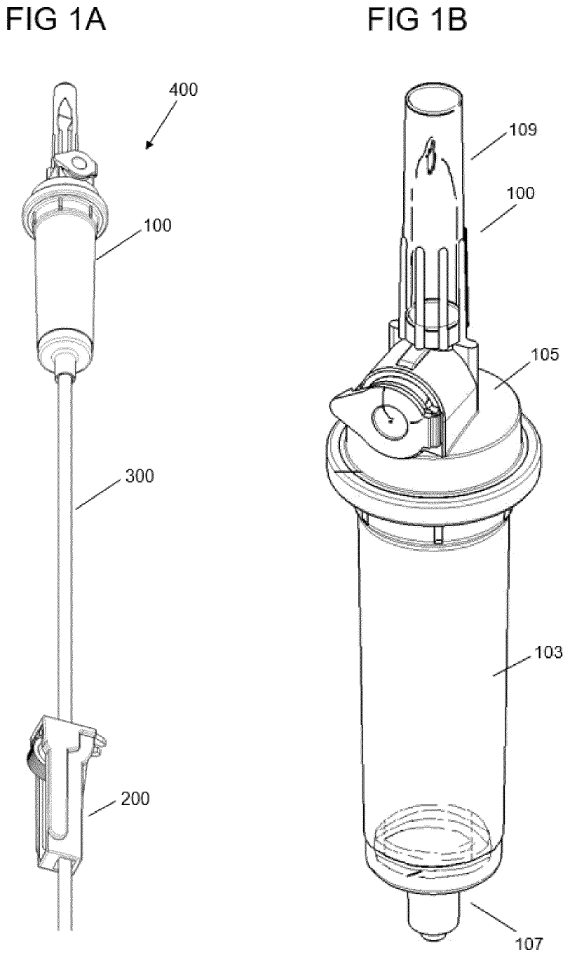

FIGS. 1A, 1B show a view of a prior art fluid administration system and a detailed view of a prior art drip chamber used in the prior art fluid administration system;

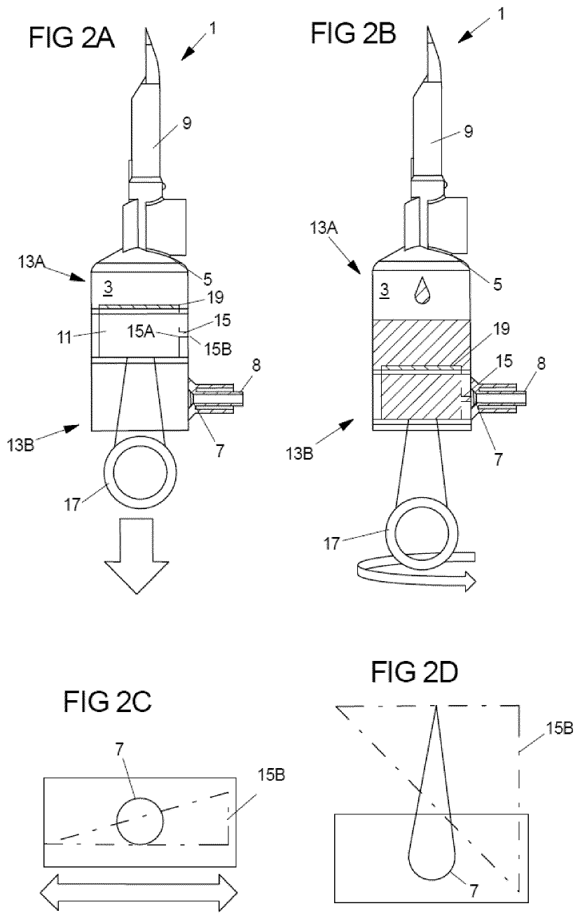

FIGS. 2A-2D show views of a drip chamber according to embodiments of the invention where the flow of fluid from the inside of the reservoir to the outlet orifice is regulated by rotating the plunger element;

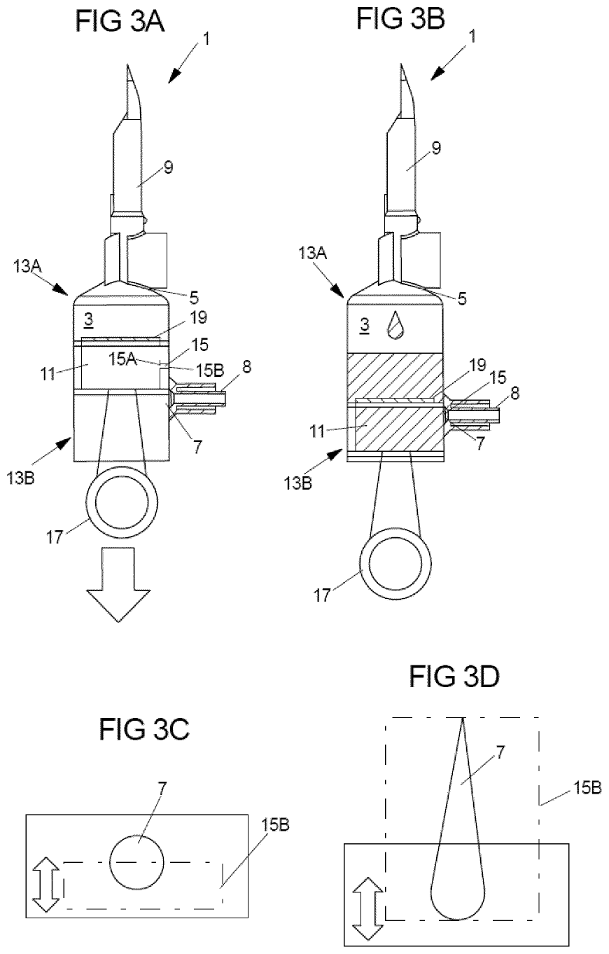

FIGS. 3A-3D show views of a drip chamber according to embodiments of the invention where the flow of fluid from the inside of the reservoir to the outlet orifice is regulated by vertically moving the plunger element along the central axis of the reservoir;

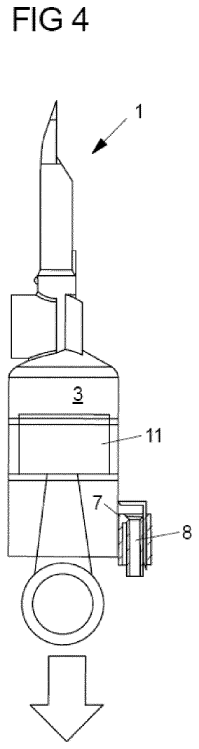

FIG. 4 shows a view of a drip chamber according to embodiments of the invention comprising a nozzle having a bent section; and

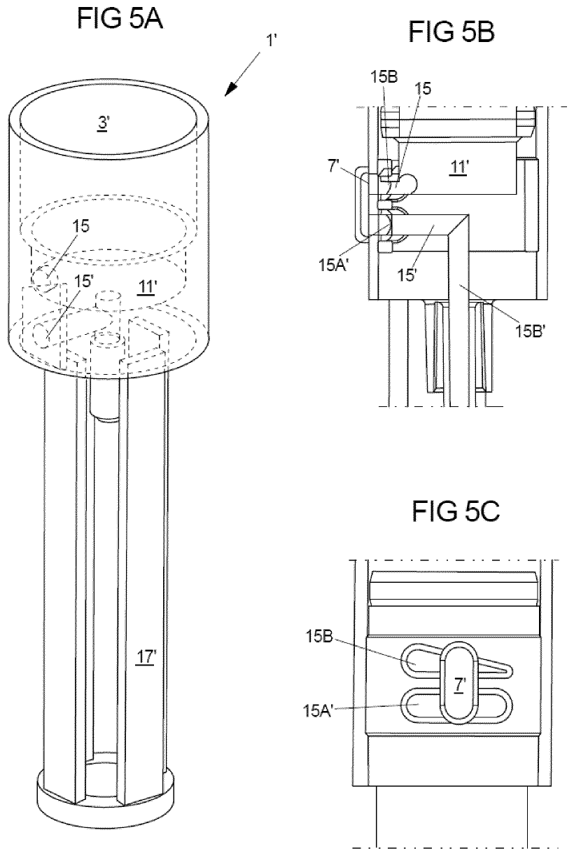

FIGS. 5A-5C show views of the lower part of a drip chamber according to an embodiment that comprises a plunger element with two fluid conducting channels.

FIG. 1A shows a fluid administration system 400 which is commonly used in the prior art, for example, for administering an IV infusion. Shown in FIG. 1A are a prior art drip chamber 100, a tube 300 connected to the drip chamber 100, through which fluid from the drip chamber 100 can flow to a patient, and a prior art clamp 200, which is shown as a so-called “roller clamp”. The clamp 200 is placed on the tube 300 for controlling the fluid flow to the patient.

In FIG. 1B a prior art drip chamber 100 is shown. FIG. 1B is a detailed view of the drip chamber 100 shown in FIG. 1A. The drip chamber 100 comprises a reservoir 103 with an inlet orifice 105 at a top end section for inserting a fluid, and an outlet orifice 107 at a bottom end section. As shown, the bottom end section is located in gravitational direction opposite and below the top end section when the drip chamber 100 is used as intended so that the gravitational acceleration is the driving force to feed the fluid from the inlet orifice 105 to the outlet orifice 107.

Also, shown in FIG. 1B is a spike 109 arranged at the inlet orifice 105 for puncturing a fluid bag (not shown) to allow fluid flowing from the fluid bag towards the inside of the reservoir 103. The sidewalls of the reservoir 103 are made from a soft plastic material so that a user can squeeze the reservoir for priming to pump fluid from the fluid bag into the reservoir 103 of the drip chamber 100.

However, as already explained herein, the prior art drip chamber 100 requires the user to squeeze the reservoir 103 several times for filling it with a fluid, which makes priming a rather cumbersome and imprecise process. Also, the prior art clamp 200 is not suitable for high precision flow regulation.

FIGS. 2A-2D show views of a drip chamber 1 according to embodiments of the invention, where the flow of fluid from the inside of the reservoir 3 to the outlet orifice 7 is regulated by rotating the plunger element 11.

In FIGS. 2A and 2B the drip chamber 1 is shown comprising the reservoir 3 with the inlet orifice 5 at a top end region 13A for inserting a fluid, and the outlet orifice 7 at a bottom end region 13B arranged in the sidewall of the reservoir 3. A nozzle 8 is arranged at the outlet orifice 7 for connecting a tube thereto. Also, shown in FIGS. 2A and 2B is a spike 9 arranged at the inlet orifice 5 for puncturing a fluid bag (not shown) to allow fluid flowing from the fluid bag towards the inside of the reservoir 3.

The depicted plunger element 9 is moveably arranged within the reservoir 3 between a first position in the top end section 13A and a second position in the bottom end section 13B. FIG. 2A shows the plunger element 11 located in the first position at the top end section 13A, while FIG. 2B shows the plunger element 11 located in the second position at the bottom end section 13B.

The shown reservoir 3 has a cylindrical shape with a substantially constant circular cross section along its length, and the plunger element 11 fits tightly within the reservoir 3 and is inserted into the reservoir 3 through an open end of the reservoir 3 at the bottom end region 13B, which is located opposite to an end of the reservoir at the top end region 13A which comprises the inlet orifice 5.

The shown plunger element 11 can be linearly pulled and pushed along the inside of the reservoir 3 between the first position and the second position.

As shown in FIGS. 2A and 2B, the first position in the top end region 13A is a position where the plunger element 11 is located closer to the top end of the reservoir 3 than to the bottom end of the reservoir 3. In not shown embodiments, the plunger element 11 can abut, or nearly abut, against a wall of the reservoir 3 which could be perpendicular to the sidewall and which comprises the inlet orifice 5. The second position in the bottom end region 13B is shown as a position where the plunger element 11 is closer to the bottom end of the reservoir 3 than to the top end of the reservoir 3, and where it is at least partially aligned with the outlet orifice 7 of the reservoir 3, e.g. at the height of the outlet orifice 7.

For example, when the plunger element 11 is located in the first position in the top end region 13A, and is then linearly pulled downwards towards the second position 13B, as indicated by the arrow in FIG. 2A, the reservoir 3 can take in fluid. Fluid inside the reservoir 3 is exemplarily shown in FIG. 2B by the shaded area inside the reservoir 3. Hence, pulling downwards the plunger element 11 creates a suction to prime the drip chamber 1.

Also shown in FIGS. 2A and 2B is a driving member 17 which is realized in the shown embodiment as loop-shaped handle that is attached to the plunger element 11 for moving the plunger element 11 inside the reservoir 3.

For regulating the flow of fluid from the inside of the reservoir 3 to the outlet orifice 7 when the plunger element 11 is located in the second position, the plunger element 11 comprises a fluid conducting channel 15 that extends through a material of the plunger element 11, and that comprises an inlet port 15A opening into the reservoir 3 and an outlet port 15B opening at least partially into the outlet orifice 7, i.e. when the outlet port 15B is at least partially aligned with the outlet orifice 7. Also shown in FIGS. 2A and 2B is a fluid filter 19 that is arranged, in fluid direction, before the fluid conducting channel 15 to prevent infectious agents, such as for example undiluted or undissolved drugs or particles of plastic or steel from administration equipment, from entering the patient's circulatory system.

As shown in FIGS. 2A and 2B the plunger element 11 has an essentially U-shaped cross-section having a sidewall extending parallel to the sidewall of the reservoir 3 and that fits tightly within the sidewall of the reservoir 3.

In the shown embodiment, the fluid conducting channel 15 is essentially an opening in the sidewall of the plunger element 11 that when at least partially aligned with the outlet orifice 7 allows fluid flowing from the inside of the reservoir 3 to the outside of the reservoir 3 via the outlet orifice 7. Depending on the position of the plunger element 11 inside the reservoir 3, the passage to the outlet orifice 7 can be closed, partially opened, or fully opened by means of rotating the plunger element 11 around its own axis when arranged in the second position as indicated by the arrow in FIG. 2B.

In FIGS. 2C and 2D exemplarily cross-sections of the outlet orifice 7 and the outlet port 15B of the fluid conducting channel 15 are shown on top each other.

Here, the cross section of the outlet port 15B could be also the cross section of the inlet port 15A, and the cross section of the channel 15 connecting the ports 15A, 15B. Also, the outlet port 15B is an elongated opening extending at least partly around a circumferential surface of the plunger element 11.

FIG. 2C shows a first embodiment of regulating the flow of fluid from the inside of the reservoir 3 to the outlet orifice 7 by rotating the plunger element 11.

The cross section of the outlet orifice 7 is shown as a circular opening and the cross section of the elongated outlet port 15B that extends at least partly around the circumferential surface of the plunger element 11 is shown as a gradually increasing, or wedged shaped opening.

FIG. 2D shows a second embodiment of regulating the flow of fluid from the inside of the reservoir 3 to the outlet orifice 7 by rotating the plunger element 11.

Here, both cross sections of the outlet orifice 7 and the elongated outlet port 15B that extends at least partly around the circumferential surface of the plunger element 11 are shown as gradually increasing, or wedged shaped openings. The outlet port 15B increases in a first direction of rotating the plunger element 11, and decreases, in a second direction, which is opposite the first direction, while the outlet orifice 7 increases from the top to the bottom.

FIGS. 3A-3D show views of a drip chamber 1 according to embodiments of the invention where the flow of fluid from the inside of the reservoir 3 to the outlet orifice 7 is regulated by vertically moving the plunger element 11 along the central axis of the reservoir 3.

The features of the drip chamber 1 shown in FIGS. 3A and 3B can be the same than the features of the drip chamber 1 shown in FIGS. 2A and 2B. However, instead of regulating the flow of fluid by rotating the plunger element 11, the flow of fluid is regulated in the embodiments shown in FIGS. 3A-3D by vertically moving the plunger element 11 along the central axis of the reservoir 3 when the plunger element 11 is located in the second position. As indicated by the arrow in FIG. 3A, the drip chamber 1 is primed by pulling the plunger element 11 downwards from the first position into the second position, and then by further pulling the plunger element 11 downwards, the flow of fluid is regulated by allowing more fluid to flow as the plunger element 11 is pulled further downwards. FIGS. 3C and 3D show corresponding cross-sections of the outlet orifice 7 and the outlet port 15B of the fluid conducting channel 15.

In FIG. 3C the cross section of the outlet orifice 7 is shown as a circular opening. The cross section of the elongated outlet port 15B that extends at least partly around the circumferential surface of the plunger element 11 is shown as having a constant width. Therefore, the cross section of the elongated outlet port 15B can be also referred to as a rectangular opening.

In FIG. 3D the cross section of the outlet orifice 7 is shown as gradually increasing from the top to the bottom, or as a wedged shaped opening, and the elongated outlet port 15B that extends at least partly around the circumferential surface of the plunger element 11 is shown as rectangular opening.

FIG. 4 shows a drip chamber 1 comprising a nozzle 8 having a bent section. Here, the nozzle 8 comprises a bent section at a distal end, which is shown as a ninety degree bent section, and which runs at least parallel to the reservoir 3 to allow an easy flow of fluid from the outlet orifice 7 towards the patient.

FIGS. 5A-5C show the lower part of a drip chamber 1′ according to an embodiment that comprises a plunger element 11′ with two fluid conducting channels 15, 15′.

As shown, the further fluid conducting channel 15′ comprises a further inlet port 15A′ opening into the outlet orifice 7 and a further outlet port 15B′ opening into a surface of the plunger element 11′ directed towards the outside of the reservoir 3.

As shown in FIGS. 5A-5C, a cap or seal is arranged on the outlet orifice 7′ so that fluid from the fluid conducting channel 15 is conducted to the further fluid conducting channel 15′. At least one of the outlet port 15B of the fluid conducting channel 15, and/or the further inlet port 15A′ of the further fluid conducting channel 15′ comprises an elongated opening with an increasing width along the direction of its extension as shown in FIG. 5C. Here, the fluid flow can be regulated by rotating the plunger element 11′ in the second position as shown in FIGS. 5A-5C. The tube, through which the fluid flows to the patient, can be connected to the further outlet port 15B′ which is located in the region of the driving member 17′.

The idea underlying the invention is not limited to the embodiments described above but may be implemented in a different fashion.

### LIST OF REFERENCE NUMERALS

1, 1′ Drip Chamber
3, 3′ Reservoir
5′ Inlet Orifice
7, 7′ Outlet Orifice
8 Nozzle
9 Spike
11, 11′ Plunger Element
13A, 13B Top End Region, Bottom End Region
15, 15′ Fluid Conducting Channel
15A, 15A′, 15B, 15B′ Inlet Port, Outlet Port
17, 17′ Driving Member
19 Fluid Filter
100 Drip Chamber—State of the Art
103 Reservoir
105 Inlet Orifice
107 Outlet Orifice
109 Spike
200 Clamp—State of the Art
300 Tube—State of the Art
400 Fluid Administration System—State of the Art

1. A drip chamber for a fluid administration system, comprising:
a reservoir comprising an inlet orifice at a top end region for inserting a fluid, and an outlet orifice at a bottom end region, wherein the bottom end region is located in gravitational direction opposite and below the top end region, and
a plunger element moveably arranged within the reservoir between a first position in the top end region and a second position in the bottom end region, wherein the plunger element comprises a fluid conducting channel for regulating the flow of fluid from the inside of the reservoir to the outlet orifice when the plunger element is located in the second position.

2. The drip chamber according to claim 1, wherein the fluid conducting channel extends through a material of the plunger element, and comprises an inlet port opening into the reservoir and an outlet port opening at least partially into the outlet orifice when the outlet port is at least partially aligned with the outlet orifice in the second position.

3. The drip chamber according to claim 2, wherein the outlet port comprises an elongated opening with an increasing width, along the direction of its extension, and wherein the elongated opening extends at least partly around a circumferential surface of the plunger element, and wherein the outlet orifice comprises a circular opening, or an opening with an increasing width along the direction of its extension.

4. The drip chamber according to claim 2, wherein the outlet port comprises an opening with a constant width along the direction of its extension, and wherein the opening extends at least partly around the circumferential surface of the plunger element, and wherein the outlet orifice comprises a circular opening, or an opening with an increasing width along the direction of its extension.

5. The drip chamber according to claim 1, wherein the outlet orifice is arranged in a sidewall of the reservoir.

6. The drip chamber according to claim 1, wherein the outlet orifice comprises a nozzle for connecting a tube to the outlet orifice.

7. The drip chamber according to claim 2, wherein the plunger element comprises a further fluid conducting channel comprising a further inlet port opening into the outlet orifice and a further outlet port opening into a surface of the plunger element directed towards the outside of the reservoir, wherein the outlet orifice is adapted to direct the fluid from the fluid conducting channel to the further fluid conducting channel, and wherein at least one of the outlet port of the fluid conducting channel, and/or the further inlet port of the further fluid conducting channel comprises an elongated opening with an increasing width along the direction of its extension, and wherein the elongated opening extends at least partly around a circumferential surface of the plunger element.

8. The drip chamber according to claim 1, wherein the plunger element comprises a sealing member encircling a lateral surface of the plunger element for providing a fluid-tight seal with the sidewall of the reservoir.

9. The drip chamber according to claim 1, wherein the plunger element comprises a driving member extending through the bottom end region for manually or automatically moving the plunger element within the reservoir.

10. The drip chamber according to claim 1, wherein the plunger element comprises a fluid filter.

11. The drip chamber according to claim 1, wherein the plunger element is adapted to move:
rotationally, around its own axis within the reservoir, and/or
vertically, along a central axis of the reservoir.

12. A method to operate a drip chamber for a fluid administration system, the method comprising:
connecting a fluid container comprising a fluid to an inlet orifice at a top end region of a reservoir to allow a fluid stream inside the reservoir, wherein the reservoir further comprises an outlet orifice at a bottom end region, wherein the bottom end region is located in gravitational direction opposite and below the top end region,
moving a plunger element within the reservoir from a first position in the top end region to a second position in the bottom end region of the reservoir, wherein the plunger element comprises a fluid conducting channel for regulating the flow of fluid from the inside of the reservoir to the outlet orifice when the plunger element is located in the second position.

13. The method according to claim 12, wherein the moving the plunger element comprises moving the plunger element vertically along a central axis of the reservoir from the first position to the second position for priming the drip chamber.

14. The method according to claim 13, further comprising aligning, in the second position, an outlet port of the fluid conducting channel extending through a material of the plunger element with the outlet orifice to at least partly open a passage for fluid from an inlet port of the channel opening into the reservoir via the outlet port of the channel to the outlet orifice.

15. The method according to claim 14, wherein the aligning further comprises:
regulating a flow rate of the fluid through the outlet port by:
rotating the plunger element around its own axis, and/or
vertically moving the plunger element along the central axis of the reservoir.

16. The drip chamber according to claim 3, wherein the increasing width of the elongated opening increases gradually or stepwise, and the increasing width of the opening increases gradually or stepwise.

17. The drip chamber according to claim 4, wherein the increasing width of the opening increases gradually or stepwise.

18. The drip chamber according to claim 6, wherein the nozzle comprises a ninety degree bent section at a distal end.

19. The drip chamber according to claim 7, wherein the increasing width of the elongated opening increases gradually or stepwise.

20. The drip chamber according to claim 9, wherein the driving member is a handle.
